(19) United States
(12) Patent Application Publication
(10) Pub. No.: US 2025/0064309 A1
(43) Pub. Date: Feb. 27, 2025

(54) Strategy and technological implementations for concurrent fluorescence measurements of multiple biological parameters in behaving animals

(71) Applicants: The Board of Trustees of the Leland Stanford Junior University, Stanford, CA (US); Howard Hughes Medical Institute, Chevy Chase, MD (US)

(72) Inventors: Simon Haziza, Sunnyvale, CA (US); Radoslaw Chrapkiewicz, Sunnyvale, CA (US); Mark J. Schnitzer, Stanford, CA (US)

(21) Appl. No.: 18/943,587

(22) Filed: Nov. 11, 2024

Related U.S. Application Data
(63) Continuation-in-part of application No. 18/749,313, filed on Jun. 20, 2024.
(60) Provisional application No. 63/522,044, filed on Jun. 20, 2023.
(60) Provisional application No. 63/598,081, filed on Nov. 11, 2023.

(51) Int. Cl. A61B 1/04; G01N 21/64

(52) U.S. Cl. CPC A61B 1/043; G01N 21/6458

(57) ABSTRACT

A method for concurrently measuring multiple biological parameters in an animal includes: a) illuminating using multiple illumination sources a region-of-interest of the animal that expresses a first genetically encoded fluorescent indicators (GEFI), a second GEFI, and a long-Stokes-shift (LSS) fluorescent compound that is insensitive to the multiple biological parameters; b) concurrently detecting fluorescence signals from the first GEFI, the second GEFI, and the LSS fluorescent compound using a multi-channel fluorescence sensing optical system; and c) processing the detected fluorescence signals to determine values of the multiple biological parameters, wherein the processing reduces instrument and/or biological artifacts in the values of the multiple biological parameters. The first and second GEFIs have distinct absorption spectra, where the first GEFI absorption spectrum overlaps the LSS fluorescent compound absorption spectrum, and the second GEFI emission spectrum overlaps the emission spectrum of the LSS fluorescent compound.

### CROSS-REFERENCE TO RELATED APPLICATIONS

This application claims priority from U.S. Provisional Patent Application 63/598,081 filed Nov. 11, 2023, which is incorporated herein by reference. This application is a continuation-in-part of U.S. patent application Ser. No. 18/749,313 filed Jun. 20, 2024, which claims priority from U.S. Provisional Patent Application 63/522,044 filed Jun. 20, 2023, which are both incorporated herein by reference.

### STATEMENT OF FEDERALLY SPONSORED RESEARCH

This invention was made with Government support under contract NS104590, NS107610, and NS120822 awarded by the National Institutes of Health, and under contract DBI-1707261 awarded by the National Science Foundation. The Government has certain rights in the invention.

### FIELD OF THE INVENTION

The present invention relates generally to fluorescence measurements in animals. More specifically, it relates to methods for concurrent fluorescence measurements of multiple biological parameters in mammals.

### BACKGROUND OF THE INVENTION

Recent strides in bioengineering of genetically encoded fluorescent indicators (GEFI) are now providing biologists with hundreds of spectrally separable sensors to monitor, in living animals, a wide range of biological phenomenon (transmembrane voltage, ions, neurotransmitters, neuromodulators, opioids, pH). To significantly advance our understanding of complex, multicellular biological systems it is critically important to decipher the precise temporal relationship of many physiological processes simultaneously.

Fiber photometry, a measurement technique that aggregates fluorescence signals using a fiber optic, is a highly pervasive approach in the field of systems neuroscience to study in vivo brain tissue dynamics during ecologically relevant behavior. A complementary technique that is becoming increasingly popular in neuroscience labs is aggregate, population level fluorescence imaging of brain dynamics in head-fixed awake animals. Recent studies have used this technique to image voltage, calcium, or neuromodulator dynamics.

However, none of the state-of-the art fiber photometry devices or 1-photon epifluorescence mesoscope systems are capable of imaging more than one biological parameter at once with low biological noise. Indeed, until now there has not been a suitable method to account for various biological artifacts while imaging two spectrally orthogonal fluorescent proteins that capture two independent biological parameters. All those limitations stifle the field from gaining new and accurate insight into the inner functioning of the brain. Accurate biological interpretation of data will be achieved if, and only if, unwanted biological artifacts are precisely monitored using a robust reference channel.

### SUMMARY OF THE INVENTION

Here, we introduce a new molecular strategy that relies on long Stokes shift fluorescent compounds. We also offer two implementations of this technique, using fiber photometry and mesoscope imaging. While the former relies on a lock-in amplification technique, the latter relies on precise control over the illumination activation and imaging frame acquisition. Our molecular solution can be used with any fiber photometry systems (used in freely behaving animals) or 1-photon epifluorescence imaging techniques (used in head-fixed animals or miniscope design used in freely moving animals) to probe the dynamics of multiple molecular phenomena concurrently in live animals. In the illustrative embodiment, we reduced this idea to practice by demonstrating artifact-free fluorescent sensing of the membrane voltage dynamic of two genetically identified neuron types in awake mice, either freely behaving using a fiber photometry system or head-restrained using a mesoscope imaging system.

This strategy is universally applicable to any fluorescent reporter compounds, from genetically encoded fluorescent proteins to synthetic dyes and nanoparticles. This strategy will reduce by 50% the cost of instrumentations used for fluorescence multiplexing of dynamic processes.

In addition to use in neuroscience labs, this technique is useful in various other areas where fluorescence sensing is used (e.g., analytical chemistry, drug screening). It could also be used for assessing the biological process dynamic of patient-derived pluripotent stem cells that recapitulate the full complexity of each patient's genetic background. An additional potential application pertains to drug discovery, as fiber optic and mesoscope systems will be able to assess the potency of a pharmacological compound on a given biological process, in vitro or in vivo.

This molecular approach provides for the first time measurement of simultaneous multiple biological parameters in a freely and head-restrained behaving animal while safeguarding against instrumental and/or biological artifacts. None of the existing technologies offers the experimental flexibility to combine multiple fluorescence sensors and monitor those signals.

For studies with two biological parameters measured in the visible range, an infrared reference fluorophore does not work because of the wavelength-dependence of hemoglobin absorption and light scattering, an infrared fluorophore poorly tracked the artifacts affecting visible fluorescence. Instead, in one implementation, we combined spectrally orthogonal green and red fluorescent proteins with a long Stokes-shift fluorescent protein with an absorption spectrum overlapping that of the green GEVI and an emission spectrum overlapping that of the red GEVI. This approach allows unambiguous detection of signals from the three fluorophores with only two illumination sources and two fluorescence detection channels. Using a NIR fluorophore would have required three illumination sources and three detection channels (and all additional opto-mechanical and electronic equipment). Our strategy reduces the overall cost of the optical system by 50%. Thus, this technology provides a cost-effective method to multiplex fluorescence signals that uses a long Stokes shift fluorescent protein to reference the biological parameter under investigation. We provide a practical implementation for fiber photometry systems that have at least two illumination sources and two photodetectors (PD, APD, PMTs or camera array sensors). We also provide a practical implementation for 1-photon epifluorescence imaging systems that have at least two illumination sources and two camera array sensors.

In one aspect, the invention provides a method for concurrently measuring multiple biological parameters in an animal, the method comprising: a) illuminating using multiple illumination sources a region-of-interest of the animal that expresses a first genetically encoded fluorescent indicators (GEFI), a second GEFI, and a long-Stokes-shift (LSS) fluorescent compound that is insensitive to the multiple biological parameters, wherein an absorption spectrum of the first GEFI is distinct from an absorption spectrum of the second GEFI, wherein the illuminating excites the first GEFI, the second GEFI, and the LSS fluorescent protein, wherein an absorption spectrum of the LSS fluorescent compound overlaps the absorption spectrum of the first GEFI, wherein an emission spectrum of the LSS fluorescent protein overlaps with an emission spectrum of the second GEFI; b) concurrently detecting fluorescence signals from the first GEFI, the second GEFI, and the LSS fluorescent compound using a multi-channel fluorescence sensing optical system; and c) processing the detected fluorescence signals to determine values of the multiple biological parameters, wherein the processing reduces instrument and/or biological artifacts in the values of the multiple biological parameters.

The multiple biological parameters may comprise transmembrane potentials of different cell-types of a brain, using genetically encoded voltage indicators (GEVIs). The LSS fluorescent compound may be, for example, a genetically-encoded protein, a dye or a nanoparticle.

In some embodiments, illuminating using the multiple illumination sources comprises illuminating using two light sources that are amplitude modulated, and wherein concurrently detecting fluorescence signals uses phase-sensitive detection. In some embodiments, illuminating using the multiple illumination sources comprises illuminating using two light sources that are sequentially activated, and wherein concurrently detecting fluorescence signals uses array detectors.

In some embodiments, the processing comprises filtering instrumental and biological noise sources, for example, using linear regression, convolutional filtering, independent component analysis, or singular value decomposition.

In some embodiments, the multi-channel fluorescence sensing optical system comprises a fiber photometry system. Concurrently detecting the fluorescence signals may comprise aggregating the fluorescence signals via an optical fiber positioned in the region of interest. The fiber photometry system may comprise two illumination sources and two detectors. Concurrently detecting fluorescence signals may use phase-sensitive detection. The fiber photometry system may comprise two illumination sources and two sCMOS array detectors. The fiber photometry system may comprise two illumination sources, wherein illuminating using multiple illumination sources comprises modulating the two illumination sources at two different frequencies. The fiber photometry system may comprise APD detectors.

In some embodiments, the multi-channel fluorescence sensing optical system comprises a 1-photon epifluorescence mesoscope, and wherein concurrently detecting the fluorescence signals comprises measuring spatiotemporal dynamics of multiple signals.

In some embodiments, the multi-channel fluorescence sensing optical system comprises a microscope imaging device. The microscope imaging device may comprise two illumination sources and two sCMOS array detectors. The microscope imaging device may comprise two light sources having two different emission wavelengths, wherein illuminating using multiple illumination sources comprises modulating the two light sources in pulsed mode to emit signals interleaved in time. Illuminating using multiple illumination sources may comprise intensity modulating signals from the multiple illumination sources, wherein the microscope imaging device comprises two sCMOS cameras, wherein detecting the multiple fluorescence signals comprises operating the two sCMOS cameras in rolling shutter mode synchronized with the intensity modulated signals from the multiple illumination sources.

### BRIEF DESCRIPTION OF THE DRAWINGS

FIG. 1A shows an overview of the main steps of a method for concurrently measuring multiple biological parameters in an animal, according to an embodiment of the invention.

FIG. 1B shows absorption and emission spectra of three fluorophores excited by two wavelength channels, according to an embodiment of the invention.

FIG. 1C illustrates a circuit for phase-sensitive frequency demodulation to isolate signals conveying the fluorescence emission powers, according to an embodiment of the invention.

FIG. 1D is a timing diagram illustrating interleaved LED illumination pulse sequences and camera detection sequences, according to an embodiment of the invention.

FIG. 1E is a schematic representation of an apparatus for implementing the method of the invention.

FIG. 2 is a schematic diagram illustrating an apparatus for fiber optic-based fluorescence sensing for implementing the method of the invention.

FIG. 3A is a schematic diagram illustrating a mesoscope fluorescence imaging apparatus for implementing the method of the invention.

FIG. 3B is a schematic diagram illustrating a timing protocol used for dual neuron-type recordings, according to an embodiment of the invention.

FIG. 3C illustrates an example of voltage and non-voltage signal components and a general post-processing scheme applied pixel-wise across a camera's field of view, according to an embodiment of the invention.

FIG. 3D illustrates clock signals corresponding used for two different embodiments of the imaging system, which detail the timing of exposure, triggering, and LED activation for dual-camera configurations, according to an embodiment of the invention.

FIG. 3E is a circuit block diagram for implementing an unmixing strategy, according to an embodiment of the invention.

FIG. 3F is a schematic diagram illustrating the effects on signals of linear filter estimation and convolution filtering steps shown in FIG. 3E.

### DETAILED DESCRIPTION OF THE INVENTION

To enable studies that dissect how different neuron classes contribute to high-frequency oscillations and waves, here we disclose techniques based on the Transmembrane Electrical Measurement Performed Optically (TEMPO) approach which captures the aggregate, population-level voltage dynamics of specific neuron-types in behaving mammals. The motivation for such measurements is that, just as in electrophysiology, optical studies of neural population dynamics can reveal collective activity patterns that may not be discernible from single-cell activity traces.

TEMPO has four basic ingredients: (1) Use of one or more GEVIs to track voltage activity in chosen neuron-types, which may be targeted using viruses, transgenic animals, or other means of selective labeling; (2) Use of a reference fluorophore that is not voltage-sensitive to track hemodynamic, brain motion and other artifacts that impact the detection of voltage-sensitive fluorescence signals; (3) Use of a dual-color fluorescence measurement apparatus; and (4) Computational unmixing of the artifactual signals from the records of neural voltage activity.

The molecular strategy of the present invention is compatible with any dual-channel optical instrument comprising two independent pairs of light sources and detectors. We illustrate the method as embodied in two instruments: an ultrasensitive form of fiber-optic TEMPO (uSMAART) for use in freely moving animals and a TEMPO mesoscope for voltage-imaging over tissue areas in head-fixed behaving animals. Both instruments can track high-frequency neural voltage oscillations (up to ˜100 Hz) without trial-averaging. Other dual-channel optical instruments can be used, such as camera-based fiber photometry systems or one-photon miniaturized microscope for studies in freely moving animals. We also describe a method relying on convolutional filtering to unmix biological artifacts and noise from voltage signals while preserving the fidelity of photon shot noise-limited, high-frequency voltage dynamics. Other unmixing algorithms can be used, such as linear regression, independent component analysis or singular value decomposition.

The molecular strategy, implemented with uSMAART, TEMPO mesoscope, or other suitable instrument, is compatible with any genetically-encoded fluorescent indicators beyond GEVIs, such as Ca²⁺, neuromodulator, neuropeptide or neurotransmitter indicators. While Ca²⁺-related fluorescence signals usually have high dynamic range, neuromodulator and neuropeptide indicators often produce smaller signals; thus, studies with the latter should benefit greatly from the instruments and unmixing methods introduced here.

TEMPO measurements involve cell-type specific expression of one or more GEVIs, a reference fluorophore to track biological and instrumentation artifacts, a dual-channel fluorescence sensing apparatus, and analytics to unmix artifact and GEVI signals. This provides voltage-sensing and -imaging systems capable of detecting high-frequency voltage dynamics of targeted neural populations in freely moving and head-fixed behaving animals, and in real-time without trial-averaging. This yielded the first recordings of beta and gamma rhythms in genetically targeted neuron-types, opening the door to mechanistic dissections of different cell-types' contributions to the generation of these oscillations.

A key facet of our TEMPO technique is the capability for dual cell-type recordings. Studies of excitatory (E) and inhibitory (I) neurons via electrical recordings often rely on classifying cell-types by their distinct spike rates, action potential waveforms, and refractory periods. However, differentiating neural subclasses (e.g. subtypes of inhibitory cells) is usually infeasible with electrical recordings alone. Although subtype identification during extracellular electrode recordings can be done via optogenetic activation, this method neither generalizes easily to more than one cell-type at once nor reports subthreshold voltage dynamics. TEMPO measurements offer these benefits and can thereby yield unique insights.

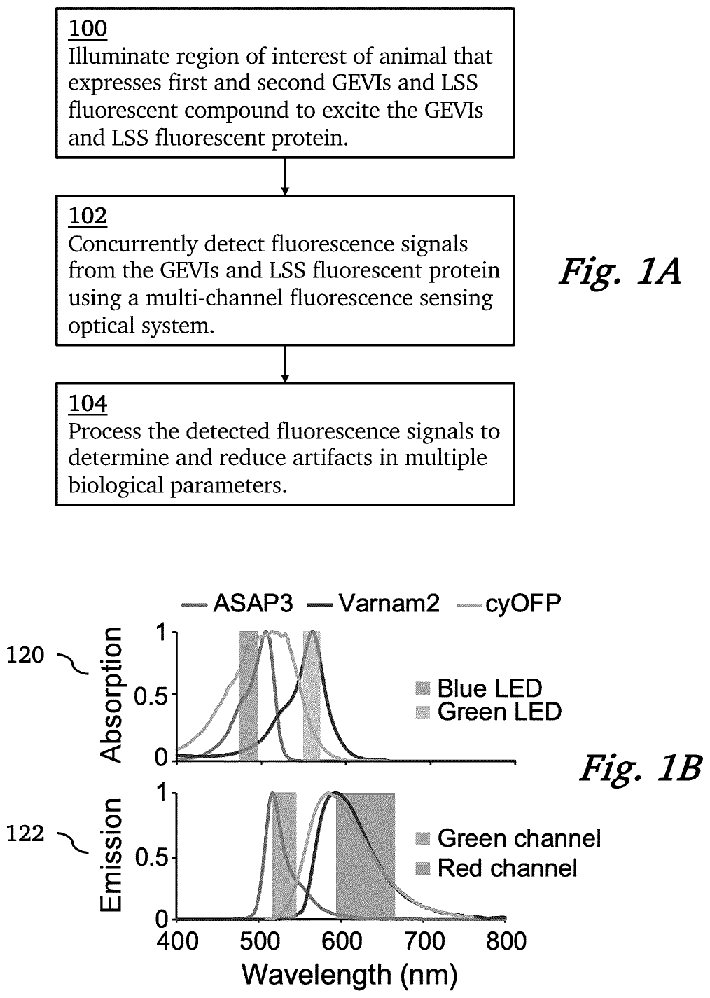

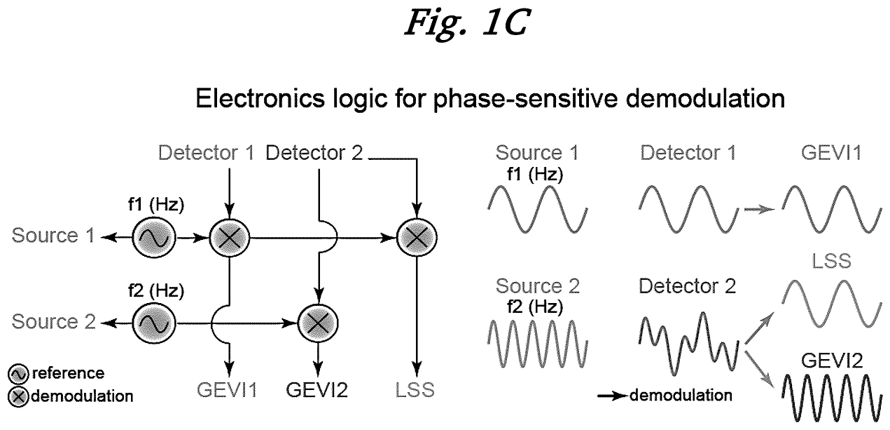

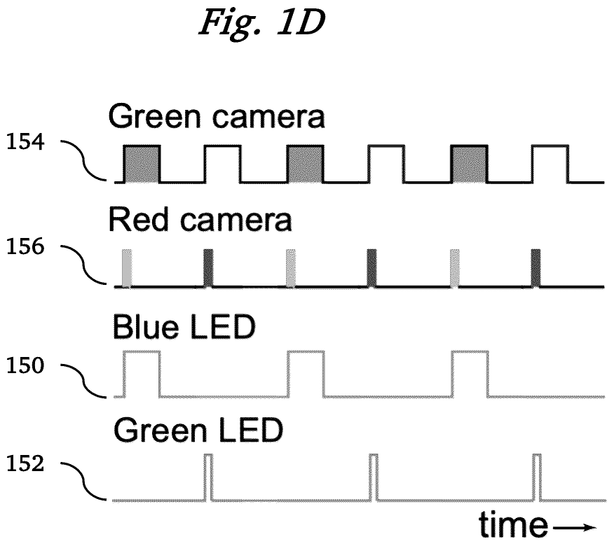

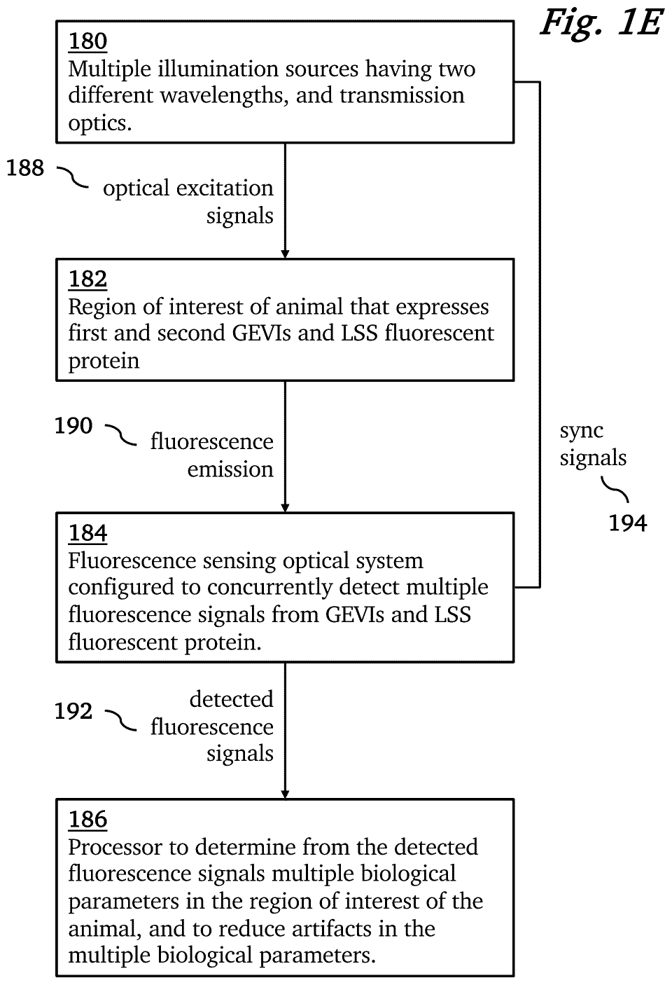

To enable concurrent measurement of multiple cell-types, TEMPO uses a spectrally separable, non-functional fluorophore as a reference. For dual cell-type TEMPO, we created a new reference approach using a long Stokes-shift fluorophore. FIG. 1A shows an overview of the main steps of a method for concurrently measuring multiple biological parameters in an animal, according to an embodiment of the invention. The multiple biological parameters may comprise, for example, transmembrane potentials of different cell-types of a brain, using genetically encoded voltage indicators (GEVIs). It will be appreciated by those skilled in the art that different types of sensors can be used to monitor the same or different biological parameters. Similarly, different reference LSS fluorescent compounds that are insensitive to the biological parameter under investigation can be used to monitor the same or different artifacts.

The expression of the fluorophores to specific cell-types is achieved using various known methods for introducing them into target tissues, such as using viral transduction or genetically modified transgenic animals that recapitulate the labeling strategy. For example, retro-orbital injection of three PHP.eB adeno-associated viruses into PV-Cre mice enables expression of Cre-dependent ASAP3 in PV interneurons, Varnam2 in pyramidal cells, and cyOFP (a reference fluorophore) in all neuron-types. The tuning of the viral titers ensures balanced fluorescence signals.

In step 100, multiple illumination sources are used to illuminate a region-of-interest of the animal that expresses a first genetically encoded fluorescent indicators (GEFI), a second GEFI, and a long-Stokes-shift (LSS) fluorescent protein that is insensitive to the multiple biological parameters. The LSS fluorescent protein may be, for example, cyOFP. The illuminating excites the first GEFI, the second GEFI, and the LSS fluorescent protein. In step 102, fluorescence signals from the first GEFI, the second GEFI, and the LSS fluorescent protein are concurrently detected using a multi-channel fluorescence sensing optical system. In step 104, the detected fluorescence signals are processed to determine values of the multiple biological parameters. The processing reduces instrument and/or biological artifacts in the values of the multiple biological parameters.

A key feature of the approach is the selection of the absorption and emission spectra of the GEFIs and the LSS fluorescent protein. In particular, the absorption spectrum of the first GEFI is distinct from an absorption spectrum of the second GEFI, wherein an absorption spectrum of the LSS fluorescent protein overlaps the absorption spectrum of the first GEFI, wherein an emission spectrum of the LSS fluorescent protein overlaps with an emission spectrum of the second GEFI. Here, two spectra are considered distinct if the wavelengths where each spectrum exceeds 50% of its maximum intensity do not overlap. Conversely, two spectra are considered overlapping if there are any common wavelengths where both spectra exceed 50% of their respective maximum intensities. However, to further prevent cross-talk in multiplexed measurements, a more conservative threshold higher than 50% would guarantee a more precise spectral separation. As an illustration, FIG. 1B shows absorption and emission spectra of three fluorophores excited by two wavelength channels as observed with TEMPO imaging. The figure shows graphs of absorption 120 and emission 122 fluorescence spectra for the three fluorophores (ASAP3, Varnam2, cyOFP). The absorption graph 120 also shows the emission spectra of the blue and green light-emitting diodes (LEDs). The emission graph 122 also shows green and red channel passbands of the emission filters in the TEMPO mesoscope. As the figures show, the ASAP3 and Varnam2 GEFIs have distinct absorption and emission spectra. The LSS fluorescent protein cyOFP has an absorption spectrum that overlaps with ASAP3 but an emission spectrum that overlaps with Varnam2.

FIG. 1C shows the electronics for phase-sensitive frequency demodulation to isolate signals conveying the fluorescence emission powers. In the dual cell-type voltage-sensing configuration, cyOFP emissions are captured on APD2 but demodulated at the same frequency as that used to modulate the 488-nm laser, while ASAP3 and Varnam2 emission signals are captured on APD1 and ADP2 respectively and demodulated at the same frequency as that used for the 488 nm and 561 nm lasers, respectively

FIG. 1D is a timing diagram illustrating the interleaved LED illumination pulse sequences 150, 152 for the blue LED 150 and green LED, respectively (i.e., alternating blue and green pulses). Also shown are corresponding detection sequences 154, 156 by the green and red cameras, respectively. The example timing protocol shown was used for dual neuron-type recordings. The durations of image frame acquisitions by the two cameras are depicted via digital voltage pulses in the top two traces 154, 156. Shading within alternate voltage pulses denotes acquired fluorescence signals. Periods of LED illumination are depicted via the voltage pulses in the bottom two traces 150, 152. When the blue LED emits a light pulse, the green camera captures ASAP3 fluorescence and the red camera captures cyOFP fluorescence. When the green LED emits a light pulse, the red camera captures Varnam2 fluorescence. This scheme allows the three different fluorescence signals to be captured unambiguously using only two illumination and two detection pathways.

The method of the invention may be implemented in various types of devices. A schematic representation of a device for implementing the method of the invention is illustrated in FIG. 1E. The device includes multiple illumination sources and transmission optics 180 which produce multiple optical excitation signals 188 of different wavelengths and transmit the signals to a region of interest 182 of an animal that expresses first and second GEVIs and an LSS fluorescent protein. The optical signals excite the first and second GEVIs and an LSS fluorescent protein, causing them to emit fluorescence light 190 which is detected by a fluorescence sensing optical system 184 configured to concurrently detect the multiple fluorescence signals from GEVIs and LSS fluorescent protein using multiple detectors for detecting different wavelengths of fluorescent light. A processor 186 processes the detected fluorescence signals 192 to determine values of multiple biological parameters in the region of interest of the animal, and to reduce artifacts in the multiple biological parameters. Synchronization signals 194 between the illumination sources and sensing optical system are used to assist with unmixing and demodulation of signals.

For illustrative purposes, we now will describe in detail two specific instruments which implement the method of the invention. In one illustrative embodiment, the method of the invention is implemented in a fiber-optic photometry apparatus, called ultra-Sensitive Measurement of Aggregate Activity in Restricted cell-Types (uSMAART). The other illustrative apparatus is a TEMPO mesoscope to image the population voltage dynamics of two neuron-types at once in head-restrained mice.

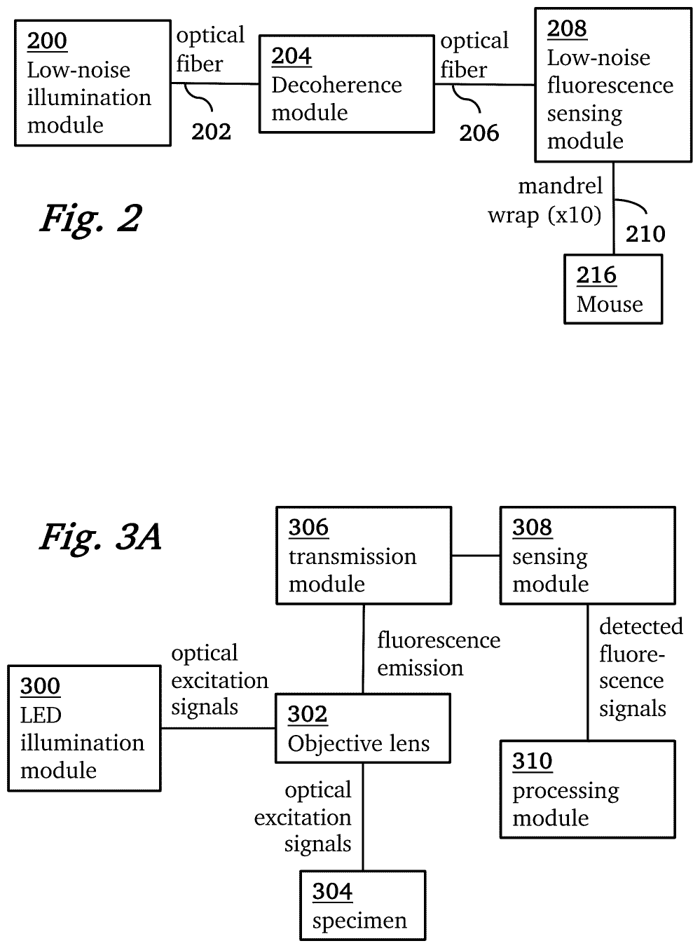

FIG. 2 is a schematic diagram illustrating an apparatus for fiber optic-based fluorescence sensing according to an embodiment of the invention. A low-noise illumination module 200 includes laser illumination sources adapted to generate green and blue light for exciting fluorescence at a modulation frequency of at least 10 kHz, or more preferably at least 100 kHz; with a laser light intensity fluctuation of at least −100 dB noise floor (or 0.01% integrated noise) across DC-200 Hz, or more preferably at least −120 dB (or 0.001%).

A decoherence module 204 coupled to the illumination module 200 via optical fiber 202 has illumination path optical elements that transmit the light. The decoherence module including an axicon lens to homogenize photon density and directionality, a dual static/dynamic diffuser to reduce speckle noise by dynamically diffusing the laser beam using a circular oscillation of the diffuser in x-direction and y-direction, and an FC/PC (physical contact fiber connector). The decoherence module 204 is coupled via an optical fiber 206 to a low-noise fluorescence sensing module 208 that transmits the illumination light into a mandrel wrap 210 flexible optical fiber for propagating the excitation photons into an animal 216 such as a mouse. The flexible optical fiber 210 also receives the emission photons from fluorescent indicators excited by the illumination light and transmits the emission light back to the low-noise fluorescence sensing module 208. The flexible optical fiber 210 has an autofluorescence signal of less than 10 pW, or more preferably less than 1 pW. The low-noise fluorescence sensing module 208 includes detection path optical elements, and a photodetector adapted to detect the fluorescence signal at the modulation frequency. A signal processor is electrically connected to the photodetector and is adapted to process signals indicative of the detected fluorescence signal.

The illumination module 200 includes blue and green solid state lasers modulated at distinct frequencies (50 and 75 kHz, respectively) to generate excitation light signals which pass through two Faraday isolators and are then split by 90:10 beam splitters to allow them to be individually monitored with photodiodes. The main portions of the light signals are combined in dichroic mirror (DM) to form a combined beam that is directed through a lens to couple the light into a fiber connector.

The excitation light from the illumination module enters the decoherence module 204 where it passes through an axicon lens and a free-space, dual-stage optical diffuser before it is coupled back into an optical fiber. The decoherence module breaks the coherence between light in different spatial modes, rendering the illumination pattern and power impervious to fiber motion.

The fluorescence sensing module 208 includes a pair of avalanche photodiodes (APDs), a combination of dichroic mirrors (DMs), and bandpass filters (BPFs) that reduce crosstalk between fluorescence channels. One dichroic mirror allows the excitation light to pass through the module into a mandrel wrap, while directing the sensed fluorescence light to two detectors. The fluorescence collection pathway has two spectral detection channels, and a single-band dichroic mirror and two bandpass filters to separate and steer green and red signals to two separate photodetectors.

A pair of lock-in amplifiers (LIA) were used for the two illumination paths to provide phase-sensitive demodulation. We amplitude-modulated the 488 nm and 561 nm lasers with analogue sinusoidal oscillations at 75 kHz and 50 kHz respectively, with 0 V to 4 V peak-to-peak amplitudes. One LIA is used to demodulate signals from the photodiode measuring 488 nm laser power fluctuations and from the APD measuring green fluorescence signals. The other LIA was used to demodulate signals from the photodiode measuring 561 nm laser power fluctuations and from the APD detecting the red fluorescence. Signals were demodulated using a linear-phase finite impulse response (FIR) filter, which is specifically designed for digital signal processing applications and allows low-pass filtering of signals with a frequency range that depends on the demodulation frequency. The low-pass filter used for demodulation was set to have a 0.1 ms time-constant and 48 dB/octave roll-off, enabling a measurement bandwidth of 0-500 Hz.

The raw, unfiltered data was analyzed by first applying a notch bandstop filter to remove the artifacts induced by the dynamic diffuser from the decoherence stage. We applied this filter to the voltage and reference traces. To remove the photobleaching dynamics, we then detrended the fluorescence signals by normalizing the trace by the time-varying baseline fluorescence, F₀, as estimated using a temporal low-pass filter. Finally, to remove the contributions of non-voltage dependent artifacts and noise sources to the fluorescence voltage trace, we unmixed the voltage and reference channel traces using a convolutional unmixing algorithm. In dual cell-type TEMPO studies, prior to the convolutional unmixing step, we included a decrosstalking step to correct for fluorescence crosstalk between the demodulated cyOFP and Varnam2 time traces. To perform the decrosstalking, we regressed the demodulated cyOFP trace against the demodulated Varnam2 trace, using bandpass-filtered versions of both traces centered on the relevant frequency band (3rd-order Butterworth filter with cut-off frequencies set at 3-7 Hz for visual cortical studies and 5-9 Hz for hippocampal studies), which contains well-characterized voltage dynamics and is generally free of artifacts. We weighted the unfiltered Varnam2 trace by the value of the regression coefficient and subtracted the resultant from the unfiltered cyOFP trace, which was then used as one of the inputs to the convolutional filter. This fluorescence decrosstalking step ensures that the subsequent convolutional filtering algorithm does not unmix or alter the voltage signals within the Varnam2 trace.

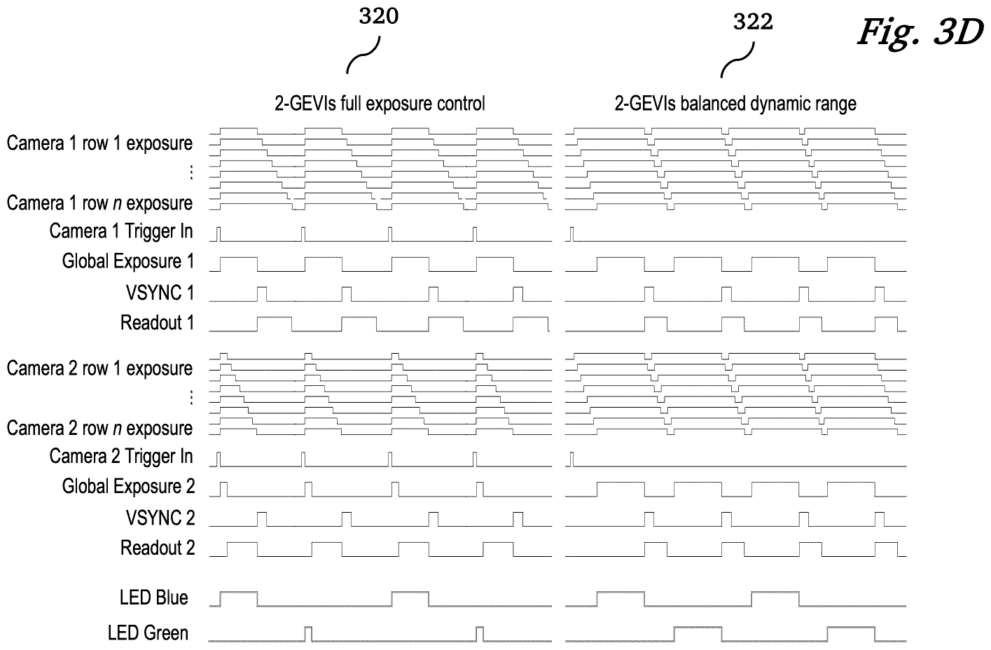

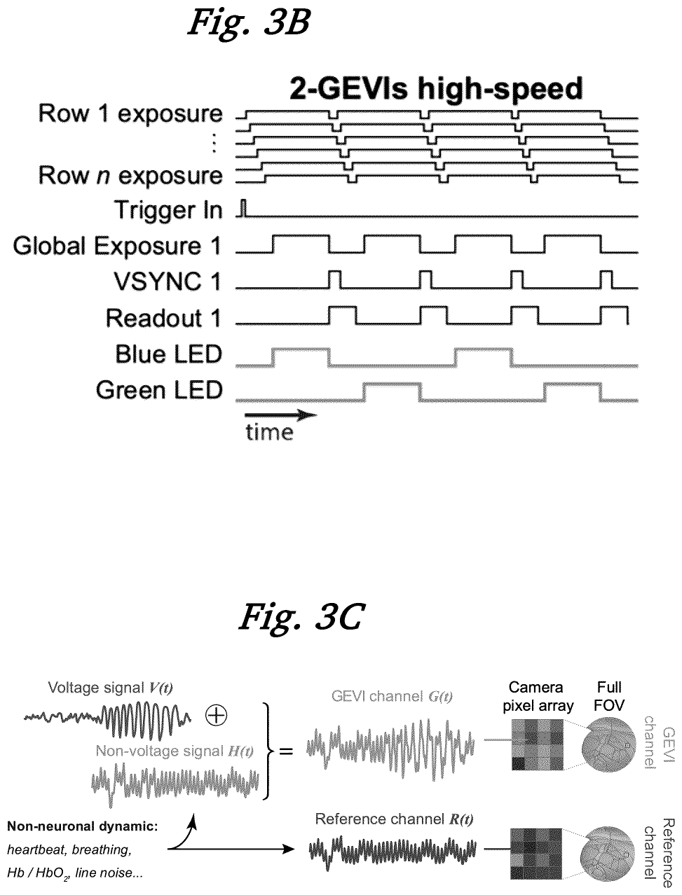

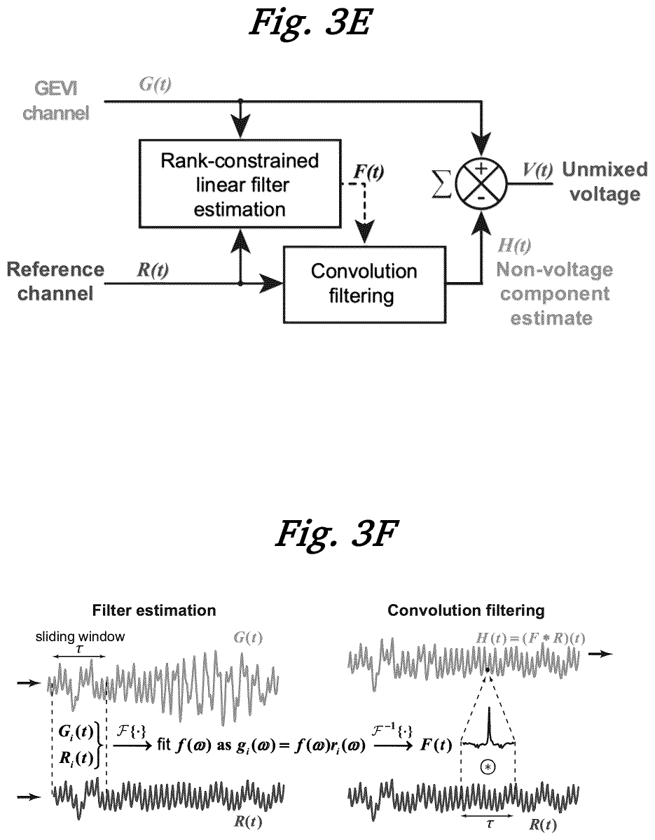

FIG. 3A is a schematic diagram illustrating a mesoscope apparatus for fluorescence imaging according to an embodiment of the invention. The apparatus is designed to image the spatiotemporal dynamics of population voltage activity in identified cell-types across the cortical surface of head-restrained mice. The apparatus produces full-frame images on the sCMOS cameras at 130 Hz, above the Nyquist frequency for neural activity in the low-gamma band (30-60 Hz). From the raw data, we unmix the voltage signals from biological and instrumentation artifacts.

A schematic diagram of a mesoscope apparatus is shown in FIG. 3A. It includes an LED illumination module 300 which generates optical excitation signals, an objective lens 302 which illuminates a specimen 304 with the excitation signals and collects resultant fluorescent emission light from a field of view of the specimen, an optical transmission module 306 that separates and provides differential focusing of two channels of the fluorescent emission light, a sensing module 308 that includes a pair of sCMOS cameras to detect images of the fluorescent light, and a processing and system control module 310.

The LED illumination module 300 uses low-noise LEDs for dual-color fluorescence excitation of two GEVIs plus a reference fluorophore in the specimen. A pair of low-noise light-emitting diodes provides two-color illumination; a corresponding pair of photodiodes monitors their emission powers for synchronization with the cameras. The illumination module provides high-power, high-stability incoherent illumination across the absorption spectrum of our green and red fluorescent GEVIs. Illumination from two LEDs (UHP-T-475-SR and UHP-T-545-SR both with low-noise and detached-fan options, Prizmatix) was combined using a dichroic beam combiner (T505LPXR, Chroma) installed in a beam combiner module (Prizmatix). The combined light then passed through a dual-bandpass clean-up filter (FF01-482_563, Semrock). The illumination then reflects off a dual-band dichroic mirror (70 mm×100 mm) into the objective lens. The dual-band dichroic (488/568-Di, Alluxa) both reflects illumination from both LEDs and transmits the fluorescence. The LED illumination module 300 has two LEDs that are pulsed and used in an alternating manner. Each LED is designed to emit optical excitation signals within specific spectral bands, tailored to match the fluorescence characteristics of the specimens being studied. Preferred emission bands for these LEDs include a range of 470-500 nm (blue) and 540-560 nm (green) to excite fluorescence within the specimen. This precise band matching enhances the detection sensitivity and specificity of fluorescence signals.

The optical excitation signals generated by the LED illumination module 300 are directed through the objective lens 302. This lens focuses the excitation light onto the specimen 304, ensuring proper illumination and maximizing the efficiency and uniformity of fluorescence excitation. The objective lens 302 also collects emitted fluorescence from the specimen and channels it towards the transmission module 306. The specimen 304 is aligned with the focal plane of the objective lens. This is where fluorescence occurs upon interaction with the optical excitation signals. The properties of the specimen influence the type and intensity of the emitted fluorescence. The objective lens 302 is a low-aberration, high numerical aperture (NA) macro-objective lens that images a field-of-view (FOV) of the specimen up to 8 mm wide (˜50 mm²). Fluorescence light returns through the objective lens, passes through the dual-band dichroic mirror and into the transmission module.

The transmission module 306 plays a critical role in directing and filtering the emitted fluorescence. It includes two dichroic mirror filters that separate fluorescence emissions based on their spectral properties. The configuration ensures that specific fluorescence wavelengths are selectively transmitted toward the sensing module 308. This aids in distinguishing between different fluorescent markers in the specimen. The optical transmission module 306 for the emission path preferably includes a 550 nm short-pass dichroic mirror placed on a super-flat substrate. The short-pass dichroic mirror (70 mm×100 mm) splits the fluorescence into two detection channels.

The sensing module 308 is composed of scientific grade sCMOS cameras and tube lenses optimized for concurrently capturing fluorescence signals. After passing through the bandpass filter, fluorescence light in each channel passes through a tube lens (85 mm effective focal length) to be focused on the cameras in the sensing module. The current embodiment employs the Orca Fusion camera paired with a Canon F1.2 USM lens. The cameras within this module are equipped with additional bandpass filters matched to the emission spectrum of the fluorescent markers being detected. This ensures that only relevant fluorescence wavelengths reach the camera sensors. The bandpass filters are a 520/41 nm for Ace-mNeon and ASAP3, and a 609/62 nm for VARNAM2. With these filters, only 0.1% of the fluorescence collected from the red fluorophore (either mRuby or Varnam) enters the green fluorescence detection channel. Whereas ˜7% of the fluorescence collected from green fluorescent ASAP3 (˜9.5% for Ace-mNeon1) enters the red fluorescence detection channel. The bandpass filters in the sensing module are designed to align with the emission properties of specific fluorophores, such as cyOFP. The red channel captures fluorescence emissions from markers like cyOFP, which are routed for processing in module 310.

The processing and system control module 310 processes and analyzes the detected fluorescence signals from the cameras to produce signals representative of multiple biological parameters of the specimen. The cameras ran in external trigger mode. Each of the two sCMOS cameras (Orca Fusion; Hamamatsu) streams image frame data to respective frame grabbers. The control module produces signals to control the camera input and output triggers as well as the LED pulsing protocol.

We precisely trigger the LED pulses within the time period when all camera rows are simultaneously illuminated. For dual cell-type TEMPO mesoscope recordings, real-time TTL pulses controlled the LEDs based on the global exposure of the cameras operating in rolling shutter mode.

The control module also generates a camera trigger signal and creates an interleaved pulse sequence that matches the camera exposure precisely. To ensure uniform illumination, the signals trigger either the green or blue LED, using every other pulse from the global exposure signal provided by the camera.

The processing module 310 processes detected fluorescence signals from the sensing module 308. It acquires and processes these signals to extract relevant information about the fluorescence characteristics of the specimen. The data analysis performed by the processing module allows for the quantification and characterization of the fluorescent markers detected within the specimen.

In operation, the imaging system begins by pulsing the LED illumination module 300 to emit excitation light. The objective lens 302 directs this light to the specimen 304, where fluorescence occurs. The emitted fluorescence is collected by the objective lens 302 and transmitted through the transmission module 306. The dichroic mirror filters within the transmission module ensure the separation of fluorescence signals based on their wavelengths. The filtered fluorescence signals are captured by the sensing module 308, which uses cameras equipped with bandpass filters to isolate specific fluorescence bands. The processing module 310 then receives these detected signals and processes them to generate data, which can be used for analysis and further applications. The emission bands of the LEDs (e.g. 470-490 nm blue, 540-560 nm green) and the bandpass filters in the transmission and sensing modules are crucial for matching with the respective fluorophore's excitation and emission spectra. The use of high-sensitivity cameras (Orca Fusion) and high-aperture lenses (Canon F1.2 USM) allows for precise and efficient capture of low-light fluorescence signals. The red channel in the sensing module is specifically adapted to capture fluorescence from markers like mRuby and cyOFP, ensuring that these markers' emission spectra are effectively processed.

FIG. 3B is a schematic of the timing protocol used for dual neuron-type recordings. To capture sufficient fluorescence light in this modality, we extend the exposure time of both cameras beyond their default values while staying within the global exposure period. Using global exposures of both cameras, we created interleaved pulses of blue and green LED illumination. The interleaved pulses allowed us to separate the signals from each GEVI using a long-Stokes shift reference fluorophore (cyOFP).

To track the emissions of three different fluorophores, we created a protocol based on the interleaving of illumination from the two different LEDs and our use of two spectrally distinct GEVIs plus a long Stokes-shift reference channel (cyOFP) (FIG. 1B). We operated the two LEDs in pulsed mode, interleaving them in time, while the two cameras recorded continuously as we encoded information about which fluorophore(s) were being excited and imaged. To capture signals from three fluorophores with varying expression patterns and intensities, cameras with different exposure times may be used.

In FIG. 3B, the trigger pulses for the LEDs were synchronized to the global-exposure trigger from both cameras that marks the time period when all rows of the camera are simultaneously acquiring photons. Synchronization to the global exposure allowed us to avoid spatial artifacts that might have arisen due to temporal mismatches between two cameras operating in rolling shutter mode.

FIG. 3B illustrates the clock signals corresponding to the specific embodiment of the imaging system, showing the detailed timing of exposure and LED activation. The first block of signals, labeled Row 1 to Row n exposure, represents the exposure times of different rows within the camera, with an overlap due to the rolling shutter effect. This pattern also includes portions of readout time and applies identically to both cameras in the system. The Trigger In signal acts as the initial trigger applied simultaneously to both cameras, initiating a sequence of operations identical for both devices. The Global Exposure 1 signal, which corresponds to the Global Exposure 2 signal on the second camera, indicates the period during which all rows are uniformly exposed. The VSYNC 1 signal is synchronized to mark the end of the Row 1 exposure period. The Readout 1 signal is aligned with the logical end or the inverse of the exposure signal, indicating the timeframe for data readout from any row of the camera. The blue and green LED activation signals are synchronized with the global exposure signals and alternate with each frame, following the count of the global exposure cycle to ensure that alternating illumination occurs throughout the imaging sequence.

FIG. 3D illustrates clock signals corresponding to two different embodiments of the imaging system, shown in panels 320 and 322, which detail the timing of exposure, triggering, and LED activation for dual-camera configurations.

Panel 320 represents the “2-GEVIs full exposure control” embodiment, where the system enables full control over the exposure settings of two individual cameras, Camera 1 and Camera 2. This embodiment assumes an external triggering mechanism where each frame waits for an incoming trigger signal before initiating. Camera 1 and Camera 2 have separate exposure signals labeled as Row 1 to Row n exposure, with their respective readout periods following the exposure. The exposure signals demonstrate the rolling shutter effect, where rows are exposed sequentially with some overlap during readout. The Camera 1 Trigger In and Camera 2 Trigger In signals mark the initiation of the exposure sequence for each respective camera, ensuring synchronized operation between them. Global Exposure 1 and Global Exposure 2 signals indicate periods where all rows of each respective camera are uniformly exposed. The VSYNC 1 and VSYNC 2 signals are aligned to the end of Row 1 exposure for Cameras 1 and 2, respectively. Readout signals (Readout 1 and Readout 2) correspond to the logical end of exposure for any row and indicate when data readout occurs. The LED Blue and LED Green signals are synchronized with their respective global exposure signals, alternating illumination between frames. This alternation is controlled by an FPGA module that counts the rising edges of the Global Exposure 1 signal, although other signals can be used as long as their rising edges precede the expected LED pulse initiation. Logic can be implemented in the FPGA module to reroute and generate the Trigger In signals for comprehensive control of the sequence.

Panel 322 of FIG. 3D corresponds to the “2-GEVIs balanced dynamic range” embodiment. Elaborating on FIG. 3B, this configuration shows the detailed exposure timing for both Camera 1 and Camera 2 with the same rolling shutter effect present in panel 320, but assuming the balance in dynamic range due to the similar expression level of use fluorophores. Camera 1 exposure times, marked from Row 1 to Row n exposure, are synchronized with Camera 2 exposure periods, with both cameras receiving an identical Trigger In signal that elicits the sequence of operations. Global Exposure 1 and Global Exposure 2 signals are present, ensuring that all rows are uniformly exposed in both cameras. VSYNC 1 and VSYNC 2 signals mark the end of the initial row exposures and serve as synchronization points. The Readout 1 and Readout 2 signals correspond to the period following exposure when data is read out from each camera's sensor. The blue and green LED activation signals in panel 322 are synchronized with global exposure signals and alternate based on the frame count. This alternation is controlled by the FPGA module that counts the rising edges of Global Exposure 1, ensuring frame-based LED switching. This embodiment guarantees that the LEDs alternate every other frame for balanced illumination in dynamic imaging sequences, enabling accurate and synchronized data acquisition across both cameras.

FIG. 3C illustrates an example of voltage and non-voltage signal components and the general post-processing scheme applied pixel-wise across a camera's field of view. The voltage signal V(t) and the non-voltage signal H(t), which includes non-neuronal dynamics such as heartbeat, breathing, hemoglobin/oxyhemoglobin fluctuations, and line noise, are combined to form distinct channels for processing. The figure highlights the usage of the GEVI channel G(t), shown in green, which captures the voltage-related signals, and the reference channel R(t), shown in red, which accounts for non-voltage components. These channels are mapped to the corresponding camera pixel array for full-field imaging. This pixel-wide processing scheme applies to each pair of pixels and is performed twice in a pairwise manner for different combinations of genetically encoded voltage indicators: specifically, for ASAP3 and cyOFP, and for Varnam2 and cyOFP, in the embodiment described.

Artifact removal from GEVI traces can be achieved using several algorithms designed to isolate voltage signals from reference signals or other sources of noise. These methods include: (1) Linear regression, which estimates the correlation coefficient through least-squares optimization and subtracts the reference signal, scaled by this coefficient, from the voltage trace. This process effectively minimizes shared variance, isolating voltage-specific dynamics; (2) Independent component analysis (ICA), which separates signals by minimizing mutual information between the voltage and reference channels, thereby achieving statistical independence of sources; and (3) Singular value decomposition (SVD) or Principal Component Analysis (PCA), which identifies the primary components capturing both voltage and artifact dynamics. By projecting the data onto the component corresponding to the voltage signal, this approach preserves the voltage signal while filtering out artifact contributions. Another approach uses a convolutional filtering method to unmix artifacts in a frequency-dependent manner; this approach accounts for the variable impact of different noise sources on the GEVI channel and thereby avoids adding reference channel noise into the GEVI traces. The convolutional filtering approach is designed to remove biological and instrumentation artifacts from neural voltage signals in a frequency-dependent way. The technique first estimates a linear filter that describes the frequency-dependent manner in which artifacts in the reference channel affect the GEVI channel. It then convolves this filter with the reference channel trace to obtain an estimate of the non-voltage signals in the GEVI channel. Lastly, it subtracts this estimate from the GEVI channel trace to estimate the true voltage signals. This convolutional unmixing preserves high-frequency voltage signals, and is applicable to both fiber-optic and imaging TEMPO data.

Here, we describe the algorithm for the case in which there is one GEVI. In studies with two GEVIs, we unmixed the reference channel content from each voltage channel individually. We treat the case in which one detection channel collects fluorescence from a GEVI (e.g., ASAP3 or Varnam2) and the other collects emissions from a reference fluorophore (e.g., mRuby2 or cyOFP). The data generally have 5 notable characteristics: (1) the GEVI channel contains a mixture of neural voltage-dependent and -independent signals, whereas the reference channel only contains signals of the latter type, i.e., that are independent of neural membrane potentials and thus are artifacts for the purposes of voltage imaging; (2) the biological artifacts (e.g., hemodynamics and brain motion) and instrumentation noise (e.g., illumination fluctuations) generally have intrinsic temporal frequency signatures; (3) oscillatory hemodynamic signals are generally the artifacts of the greatest magnitude and have characteristic frequencies concentrated around that of the heartbeat and its harmonics; (4) hemodynamic oscillations are present in the same frequency bands and are generally coherent between the two channels, but do not always oscillate with the same phase in the two channels; (5) the spatial distribution of hemodynamic artifacts is non-uniform across the field-of-view.

FIG. 3E is a block diagram of the unmixing strategy. A rank-constrained linear filter, F(t), is estimated using both fluorescence channels and captures the manner in which artifacts seen in the reference channel coherently invade the voltage channel in different frequency bands. F(t) is convolved with the reference channel, R(t), to attain an estimate, H(t), of the non-voltage signals present in the GEVI channel. H(t) is subtracted from the GEVI channel trace, G(t), to attain an estimate of the true voltage signals, V(t). Plain arrows represent data flow. Dashed arrow denotes transfer of the F(t) function.

FIG. 3F is a schematic of the filter estimation and convolution steps in FIG. 3E. We used the Fourier transform F{⋅} to obtain the frequency-domain representations, r(ω) and g(ω), of R(t) and G(t), respectively, over sliding time windows of duration, τ (typically 0.5-2 s). We then used linear regression to compute for each frequency, ω, the value of the filter coefficient, f(ω), that best describes the amplitude with which the reference signal, r(ω), is present in the voltage channel, g(ω), across the set of all time windows. After performing an inverse Fourier transform of f(ω) to obtain the filter's time-domain representation, F(t), we estimated H(t) by convolving F(t) with R(t) and then subtracted H(t) from G(t) to obtain V(t).

We implemented our unmixing algorithm using the assumption that the signals in the GEVI channel, G(t), are a sum of the voltage signals, V(t), plus a voltage-independent component, H(t):

$$G ( t ) = V ( t ) + H ( t ) \qquad ( Eq . 1 )$$

Based on above observations (1-5), we formulated the problem of estimating the non-voltage signals, H(t), as a Wiener filter estimation problem in which we seek a filter, F(t) such that the convolution of the reference channel trace, R(t), with the filter, F(t), yields H(t):

$$H ( t ) = ( F * R ) ( t ) \qquad ( Eq . 2 )$$

Our physiological intuition for why this formulation makes sense is that H(t) predominantly arises from changes in the optical properties of the tissue, due to time-variations in oxygenated (HbO) and deoxygenated (Hb) hemoglobin content. These two forms of hemoglobin will have distinct influences on the GEVI and reference fluorescence signals, but these influences are not independent. A simplified way of depicting the dynamics relating the concentrations of Hb and HbO is to represent them as a linear dynamical system, which implies a linear dynamical relationship between R(t) and H(t) that can be represented as a convolution in the time-domain. With this framework, the main steps of the technique are as follows:

1. We split the traces from the GEVI, Gₖ(t), and reference channels, Rₖ(t), into N temporal segments of duration τ (typically 0.5-2 s) indexed by the variable k=0, 1, . . . N, and with fractional overlap 1−γ∈[0,1] (typically 0.75)

$${G}_{k} ( t ) = G ( t + k γτ ) , {R}_{k} ( t ) = R ( t + k γ τ ) , t ∈ [ 0 , τ ) \qquad ( Eq . 3 )$$

We compute the corresponding Fourier transforms gₖ(ω) and rₖ(ω):

$${g}_{k} ( ω ) = F \{ {G}_{k} ( t ) w ( t ) \} , {r}_{k} ( ω ) = F \{ {R}_{x} ( t ) w ( t ) \} \qquad ( Eq . 4 )$$

where F{⋅} denotes a Fourier transform and w(t) is a windowing function (e.g., a Hann window).

2. We estimate the Wiener filter in the frequency domain, f(ω), by concurrently fitting a complex coefficient fₖ(ω) for every frequency ω to approximate the GEVI channel signal gₖ(w) with the reference channel signal rₖ(ω) for all N segments:

$$f ( ω ) = \frac{{〈 {g}_{k} ( ω ) {r}_{k}^{*} ( ω ) 〉}_{k}}{{〈 {r}_{k} ( ω ) {r}_{k}^{*} ( ω ) 〉}_{k}} \qquad ( Eq . 5 )$$

where r* is the complex conjugate of r and [symbol] ⋅ [symbol] ₖ denotes averaging over the N segments. We limit the upper bound of the filter amplitude relative to the amplitude of the linear regression estimated at the heartbeat frequency ω₀, with α>0 (typically 1.1)

$${f ( {ω}^{′} \vert}_{\vert f ( {ω}^{′} ) \vert \times α \vert f ( {ω}_{0} ) \vert} ) = α \vert f ( {ω}_{0} ) \vert {e}^{targf ( {ω}^{′} )} . \qquad ( Eq . 6 )$$

If ω₀ is the fundamental harmonic of the heartbeat frequency, the amplitude |f(ω₀)| can be interpreted as a linear regression coefficient between the channels filtered at the heartbeat frequency. We set the spectral amplitude limit for the filter to α|f(ω₀)|, thus explicitly prohibiting unbounded amplification.

3. We apply an inverse Fourier transform to find the time-domain representation of the filter F(t):

$$F ( t ) = {F}^{- 1} \{ f ( ω ) \} \qquad ( Eq . 7 )$$

We convolve F(t) and R(t) in the time-domain to estimate the non-neuronal contribution H(t), (Eq. 2), and subtract H(t) from the GEVI signal G(t) to recover the unmixed voltage signal V(t):

$$V ( t ) = G ( t ) - H ( t ) \qquad ( Eq . 8 )$$

The above technique can be interpreted as a rank-constrained linear regression in the frequency domain. We fit a complex-valued scaling coefficient fₖ(ω) for every frequency ω to simultaneously approximate the voltage channel signal gₖ(ω) with the reference channel signal rₖ(ω) for all segments, k. The rank of the regression (i.e., the number of free parameters or the number of points in the filter) is equivalent to the filter length, t, and thus inversely proportional to the spectral resolution.

We determined the appropriate segment length τ (usually 1.0-1.5 s) and optimal relative spectral limit, α (1.0-1.3) by splitting the data in half into train and test segments and minimizing the remaining signal in the test movie segment after unmixing with respect to the parameters t and a. The unmixing procedure is robust with regard to minor changes in the values of these parameters. Larger segment lengths allow for greater spectral resolution of the filter, whereas smaller segment lengths result in more robust estimation in low SNR recordings.

In some recordings, the noise floor in the reference channel was higher than that in the GEVI channel. In such recordings, unmixing would lead to masking of high-frequency signals underneath a noise floor inherited from the reference channel. Therefore, we performed a pre-filtering step that involved estimating H^{avg}(t) using a spatially averaged reference movie, R^{avg}(t), and then removing H^{avg}(t) at the single pixel level:

$${V}^{res} ( t ) = G ( t ) - {H}^{avg} ( t ) \qquad ( Eq . 9 )$$

Then, we performed a second convolutional filtering at the single pixel level between signals in the reference channel, R(t), and those in the voltage channel, V^{res}(t). The pre-filtering step is reasonable because, as non-neural activity generally does not travel spatially, it boosts the SNR of R^{avg}(t). The suitable scale of spatial averaging depends on the relative noise levels between the two channels and was usually set to be ˜0.5-1.5 mm. Although more spatial averaging leads to improved SNR values, reduced spatial averaging allows the spatial variations of the hemodynamics to be captured at higher accuracy. Overall, compared to a simple (i.e., frequency independent) regression, this unmixing method better removed broad- and narrow-band artifacts and did not transfer noise from the reference to the GEVI channel, which was critical for detecting high-frequency activity with high sensitivity.

1. A method for concurrently measuring multiple biological parameters in an animal, the method comprising:
a) illuminating using multiple illumination sources a region-of-interest of the animal that expresses a first genetically encoded fluorescent indicators (GEFI), a second GEFI, and a long-Stokes-shift (LSS) fluorescent compound that is insensitive to the multiple biological parameters, wherein an absorption spectrum of the first GEFI is distinct from an absorption spectrum of the second GEFI, wherein the illuminating excites the first GEFI, the second GEFI, and the LSS fluorescent compound, wherein an absorption spectrum of the LSS fluorescent compound overlaps the absorption spectrum of the first GEFI, wherein an emission spectrum of the LSS fluorescent compound overlaps with an emission spectrum of the second GEFI;
b) concurrently detecting fluorescence signals from the first GEFI, the second GEFI, and the LSS fluorescent compound using a multi-channel fluorescence sensing optical system;
c) processing the detected fluorescence signals to determine values of the multiple biological parameters, wherein the processing reduces instrument and/or biological artifacts in the values of the multiple biological parameters.

2. The method of claim 1, wherein the multiple biological parameters comprise transmembrane potential of different cell-types of a brain, using genetically encoded voltage indicators/reporters (GEVIs) as GEFIs.

3. The method of claim 1, wherein the LSS fluorescent compound is an LSS protein, dye, or nanoparticle.

4. The method of claim 1, wherein illuminating using the multiple illumination sources comprises illuminating using two light sources that are amplitude modulated, and wherein concurrently detecting fluorescence signals uses phase-sensitive detection.

5. The method of claim 1, wherein illuminating using the multiple illumination sources comprises illuminating using two light sources that are sequentially activated, and wherein concurrently detecting fluorescence signals uses array detectors.

6. The method of claim 1, wherein the processing comprises filtering instrumental and biological noise sources using linear regression, convolutional filtering, independent component analysis, or singular value decomposition.

7. The method of claim 1, wherein the multi-channel fluorescence sensing optical system comprises a 1-photon epifluorescence microscope, and wherein concurrently detecting the fluorescence signals comprises measuring spatiotemporal dynamics of multiple signals.

8. The method of claim 1, wherein the multi-channel fluorescence sensing optical system comprises a fiber photometry system.

9. The method of claim 8, wherein concurrently detecting the fluorescence signals comprises aggregating the fluorescence signals via an optical fiber positioned in the region of interest.

10. The method of claim 8, wherein the fiber photometry system comprises two illumination sources and two detectors.

11. The method of claim 8, wherein concurrently detecting fluorescence signals uses phase-sensitive detection.

12. The method of claim 8, wherein the fiber photometry system comprises two illumination sources and two sCMOS array detectors.

13. The method of claim 8, wherein the fiber photometry system comprises two illumination sources, wherein illuminating using multiple illumination sources comprises modulating the two illumination sources at two different frequencies.

14. The method of claim 8, wherein the fiber photometry system comprises APD detectors.

15. The method of claim 1, wherein the multi-channel fluorescence sensing optical system comprises a microscope imaging device.

16. The method of claim 15, wherein the microscope imaging device comprises two illumination sources and two sCMOS array detectors.

17. The method of claim 15, wherein the microscope imaging device comprises two light sources having two different emission wavelengths, wherein illuminating using multiple illumination sources comprises modulating the two light sources in pulsed mode to emit signals interleaved in time.

18. The method of claim 15, wherein illuminating using multiple illumination sources comprises intensity modulating signals from the multiple illumination sources, wherein the microscope imaging device comprises two sCMOS cameras, wherein detecting the multiple fluorescence signals comprises operating the two sCMOS cameras in rolling shutter mode synchronized with the intensity modulated signals from the multiple illumination sources.
